(19) United States
(12) Patent Application Publication
(10) Pub. No.: US 2025/0259223 A1
(43) Pub. Date: Aug. 14, 2025

(54) Grocery Shopping Assistant

(71) Applicant: Helthy Inc., Sierra Madre, CA (US)

(72) Inventors: Lisa Yun Zhu, Sierra Madre, CA (US); Prashanth Saka, Boston, MA (US)

(21) Appl. No.: 19/052,631

(22) Filed: Feb. 13, 2025

Related U.S. Application Data
(60) Provisional application No. 63/552,857, filed on Feb. 13, 2024.

(51) Int. Cl. G06Q 30/0601; G16H 20/60

(52) U.S. Cl. CPC G06Q 30/0631; G06Q 30/0623; G06Q 30/0643; G16H 20/60

(57) ABSTRACT

In an embodiment, a method includes, responsive to a user of a user device selecting a food product in a user interface of the user device, determining nutrition information of the food product. The method further includes processing the nutrition information of the food product using a dietary model, the dietary model providing a purchase recommendation and one or more recommendation reasons. The method further includes displaying, in the user interface, the purchase recommendation. Therefore, a personalized grocery shopping assistant for people with diseases having dietary restrictions, such as prediabetes and type 2 diabetes is advantageous.

### RELATED APPLICATION(S)

This application claims the benefit of U.S. Provisional Application No. 63/552,857, filed on Feb. 13, 2024, and is related to U.S. Provisional Application No. 63/232,461, filed on Aug. 12, 2021 and U.S. Provisional Application No. 63/248,276, filed on Sep. 24, 2021. The entire teachings of the above applications are incorporated herein by reference.

### BACKGROUND

Diabetes, specifically prediabetes and type 2 diabetes, occurs when the human body has trouble using the sugar it gets from food for energy. The body builds up sugar in the bloodstream because the sugar cannot get into the cells, and this has immediate effects for the patient, such as blurry vision, and long-term effects, such as heart disease and blindness.

The most common test to identify prediabetes and type 2 diabetes is the Hemoglobin A1C test. Between 5.7 and 6.5% suggests levels are in the prediabetes range, and 6.5% or higher suggests type 2 diabetes. Patients are most often diagnosed at 40-60 years of age, first with prediabetes at their primary care physician where they receive educational materials and advice on nutrition and exercise. Physicians sometimes recommended a nutritionist; however, many patients choose not to see one due to the fear of being stigmatized or placed on an unwanted diet. Primary care physicians also diagnose patients with diabetes, and on the spot, prescribe diabetes medication. These physicians usually feel that they can manage the patient themselves until patients need to be placed on multiple drugs for other diseases, and at which point, patients are assigned to an endocrinologist.

### SUMMARY

In an embodiment, a method includes, responsive to a user of a user device selecting a food product in a user interface of the user device, determining nutrition information of the food product. The method further includes processing the nutrition information of the food product using a dietary model, the dietary model providing a purchase recommendation and one or more recommendation reasons. The method further includes displaying, in the user interface, the purchase recommendation.

In an embodiment, the method includes determining at least one alternative food product and displaying, with the purchase recommendation, the at least one alternative food product.

In an embodiment, determining nutrition information of the food product includes:
a) parsing food information displayed on a retailer website,
b) accessing a database of nutrition information of food products, or
c) interfacing with an application programming interface of the retailer website having the nutrition information.

In an embodiment, a dietary model is a rule based model that factors in the nutrition information. The dietary model may factor in nutrition information including at least one of: presence of trans-fats, amount of added sugars, amount of refined grains, amount of artificial sweeteners, amount of sodium. The dietary model may further factors in user information. The dietary model may calculate a daily limit for the user based on their information.

In an embodiment, the dietary model calculates a daily maximum of 45% total calories should come from carbs, a daily maximum of 40% of daily calories should come from fat, and a daily maximum of 30% of daily calories should come from protein.

In an embodiment, the dietary model flags products as not recommended to purchase when they have more than 55% total calories coming from carbohydrates, 50% coming from fat, and 40% from protein.

In an embodiment, the food product is at least one of a food product at a store, a meal kit, and a meal from a restaurant.

In an embodiment, the user interface is at least one of a browser with a browser extension installed, a browser connected to a retailer website, and a mobile application.

In an embodiment, a system includes a processor; and a memory with computer code instructions stored thereon. The processor and the memory, with the computer code instructions, being configured to cause the system to, responsive to a user of a user device selecting a food product in a user interface of the user device, determine nutrition information of the food product. The processor and the memory further are configured to process the nutrition information of the food product using a dietary model, the dietary model providing a purchase recommendation and one or more recommendation reasons. The processor and the memory are further configured to display, in the user interface, the purchase recommendation.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a diagram illustrating a typical journey of a diabetes patient.

FIG. 2 is a diagram illustrating an example embodiment of a grocery recommendation software to assist diabetic patients.

FIGS. 3A-D are diagrams illustrating example user interfaces of the present disclosure.

FIG. 4 is a diagram of a table or database illustrating products, and various statistics associated with each product, including glycemic load.

FIG. 5A is a flow diagram illustrating an example embodiment of setting up the extension for the user.

FIGS. 5B-I are diagrams illustrating example embodiments of the user interface setting up the extension for the user.

FIG. 6A is a flow diagram illustrating an example embodiment of using the extension.

FIGS. 6B-H are diagrams illustrating example embodiments of the user interface with a user using the extension.

FIG. 7 is a diagram illustrating a study conducted with test images, illustrating the effectiveness of the method in the real world with real patients.

FIG. 8 illustrates a computer network or similar digital processing environment in which embodiments of the present invention may be implemented.

FIG. 9 is a diagram of an example internal structure of a computer (e.g., client processor/device or server computers) in the computer system of FIG. 8.

### DETAILED DESCRIPTION

A description of example embodiments follows.

In embodiments, this disclosure descripts a personalized grocery shopping assistant for people with diseases having dietary restrictions, such as prediabetes and type 2 diabetes. The system is nicknamed “Helthy” and in other embodiments is nicknamed “Gromm,” and is referred to as such in this disclosure.

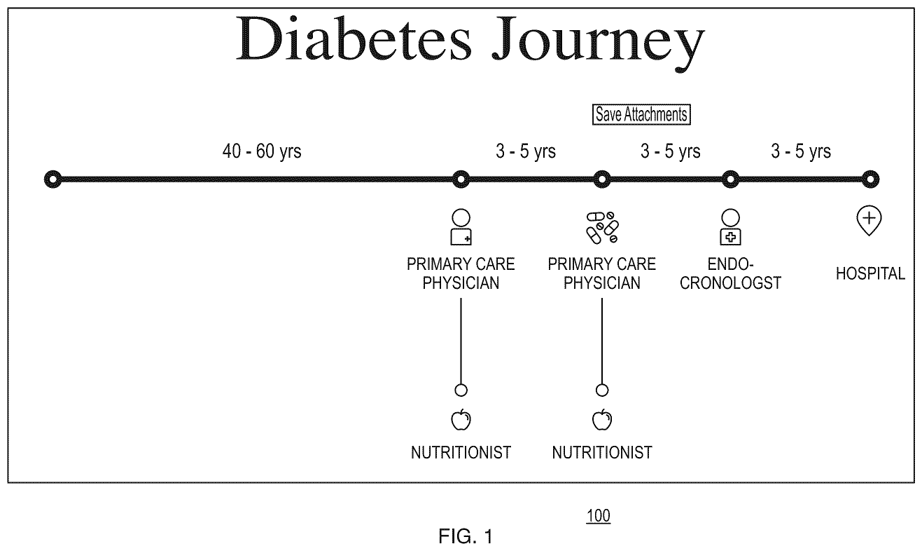

FIG. 1 is a diagram 100 illustrating a typical journey of a diabetes patient. Diabetes is a widespread disease. In 2021 in the United States of America (USA), 120 M adults have prediabetes and type 2 diabetes—one-third of the adult USA population at that time. diabetes is the most expensive chronic condition to treat in the USA; every year, the US spends about $330 billion on diabetes care.

Obesity is also a leading risk factor in diabetes. In the US, adolescent obesity is on the rise, and the CDC predicts that 1 in 3 Americans will develop type 2 diabetes sometime in their lifetime, and causing further complications down the road.

Upon diagnosis, doctors typically recommend eating healthy, exercising, and following diabetes guidelines. While this is sound advice, often the patient is on their own to make decisions and independently make decisions on what foods are best for their health. Therefore, there is a gap between clinical nutrition advice and dietary lifestyle changes. While nutritionists are one option, there are a limited number of nutritionists, and some patients do not have time to make extra appointments. In addition, some patients are fearful of another person performing meal planning and telling them what to eat. In addition, as most diabetics are diagnosed after age 40, they have decades of eating and food shopping habits to break that can be difficult for nutritionist alone.

Bridging the gap between medical advice and dietary lifestyle change upon diagnosis therefore can help ease the burden of stakeholders including nutritionists, primary care physicians, patients, and healthcare investors. Bridging such a gap will help patients and the healthcare system as a whole manage and, in some cases, potentially reverse diabetes.

While the present system can be used with anyone diagnosed with diabetes, for the above reasons, it can be most helpful to employ lifestyle change with people 35+ years of age and older with prediabetes or Type 2 diabetes in the US. A target user can be a patient with blood glucose being between 5.7%-7.9%, have a length of diagnosis is between 0-10 years, and their condition can be managed without diabetes medication or insulin. The target user lives a busy lifestyle, has ingrained cultural and personal habits, and is unsure of their dietary choices post-diagnosis. They usually get nutrition information from the internet (Google™ or WebMD®) or their doctor. In addition, they are committed to managing and, possibly, reversing the disease in a fiscally responsible manner.

Other solutions to this problem are meal kits, which can be very expensive for the end user, have too rigid of a diet, and are disruptive to people who want to maintain the way they shop for groceries.

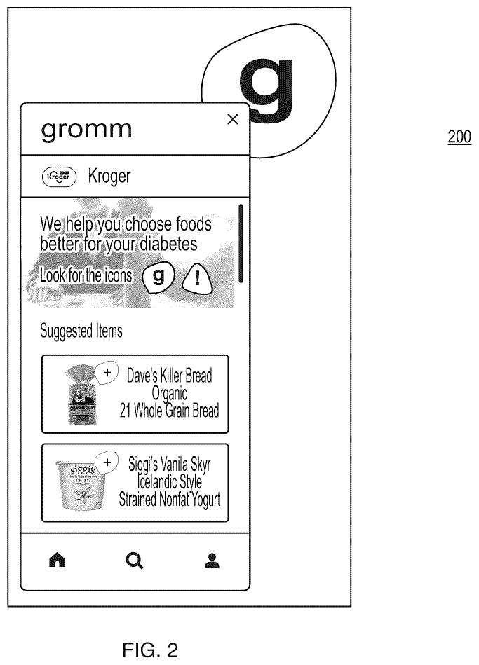

FIG. 2 is a diagram 200 illustrating an example embodiment of a grocery recommendation software to assist diabetic patients. In embodiments, software can provide real-time recommendations as people shop for groceries online. In embodiments, the software can be implemented as a browser extension. This browser extension is called Helthy. Helthy is designed to make dietary lifestyle changes actionable; specifically, the tool provides simple visual cues that overlay on product pages for users to know which products fit within their healthy eating guidelines based on glycemic loads, macronutrients, ingredients, biometrics, food preferences, and goals. The method is personalized to the goals of each user. Helthy does not require the permission of retailers or website owners, which means that Helthy is compatible across retailers so people can shop freely as they normally would—not at only group of approved websites. The diagram of FIG. 2 illustrates an example Helthy window displayed while visiting on a Kroger® website, where Helthy suggests healthy items for the diabetic patient such as Dave's Killer Bread and Siggi's Vanilla Skyr Icelandic Style Yogurt.

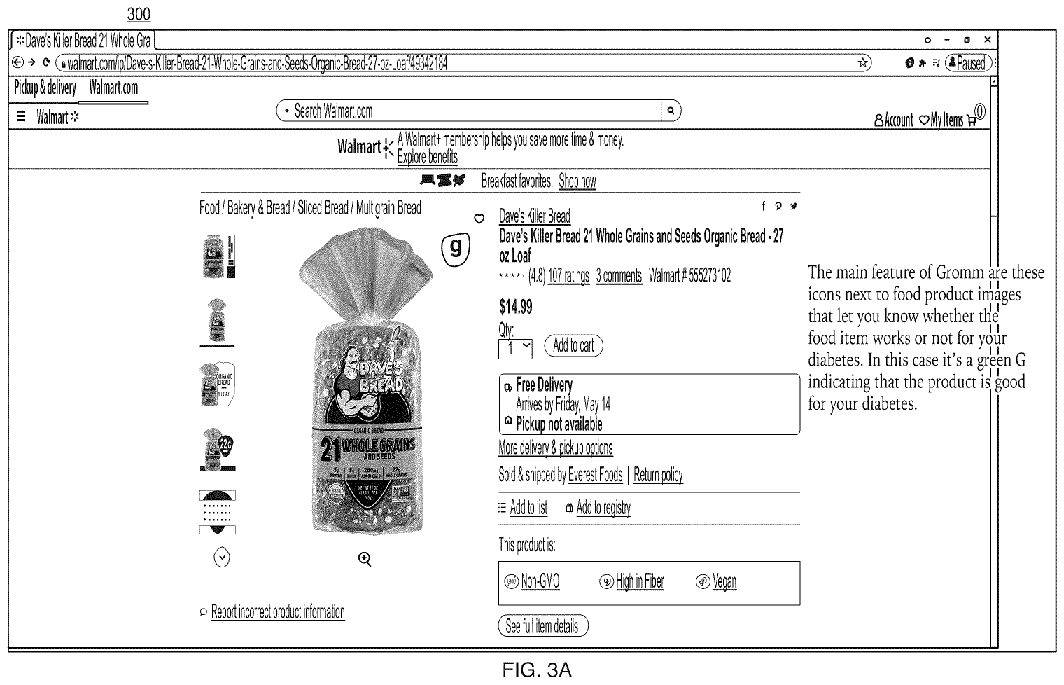

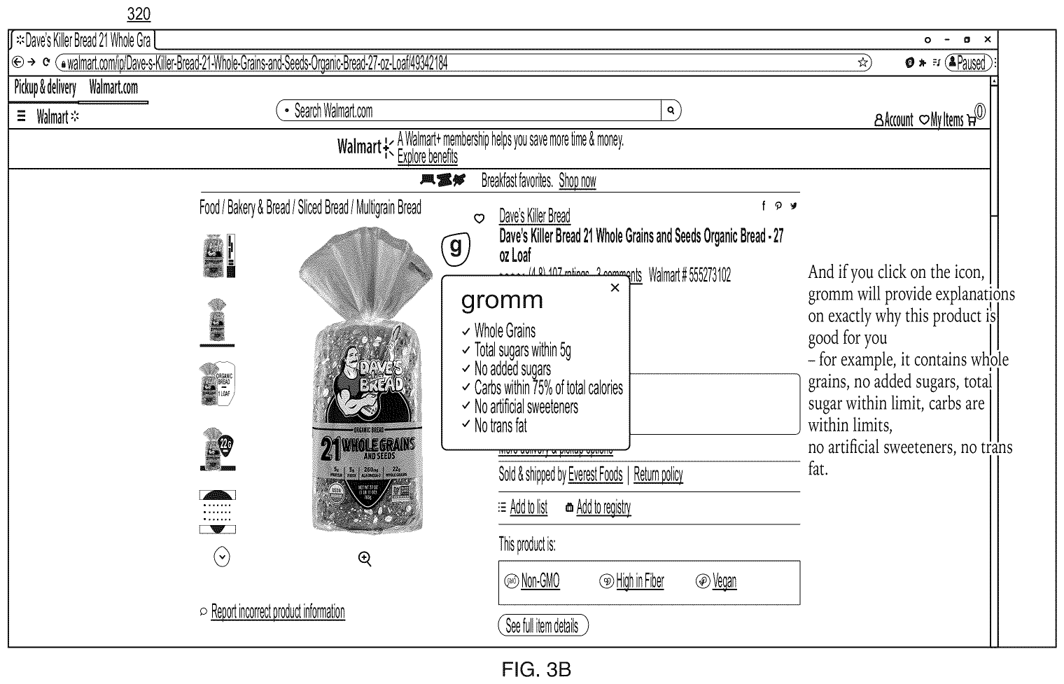

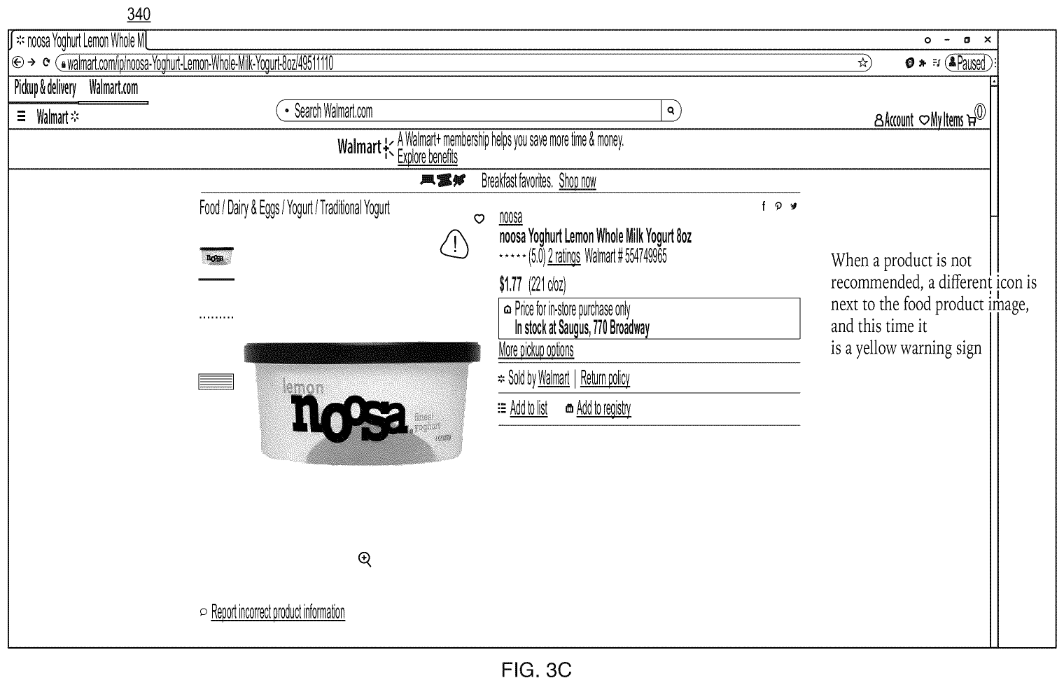

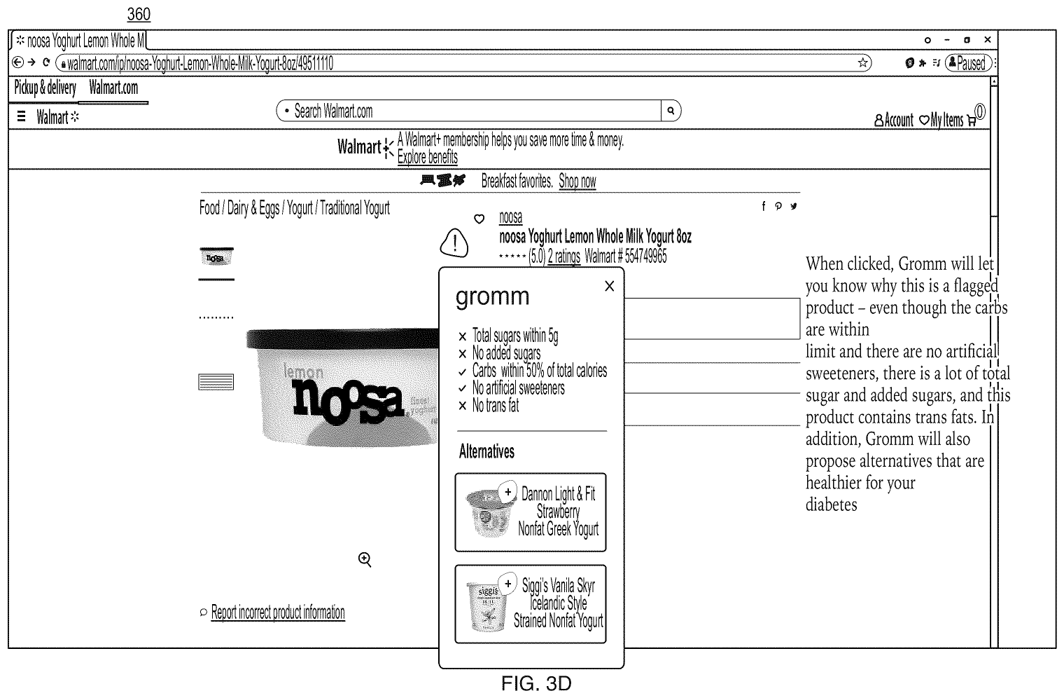

FIGS. 3A-D are diagrams 300, 320, 340, and 360 illustrating example user interfaces of the present disclosure. In a mass survey of 300 participants who either have prediabetes or type 2 diabetes, and shop for groceries online, a majority of people agreed that while shopping for groceries, they desire recommendations for their prediabetes or diabetes in real-time. A main feature of Helthy is presenting icons next to food product images that let a user know whether the food item works or not for their diabetes. In FIG. 3A, the user interface 300 illustrates a green G indicating that the product is good for the user's diabetes. When the user selects/clicks on the icon, Helthy provides explanations on why the product is good for the patient (e.g., it contains whole grains, no added sugars, total sugar within limit, carbs are within limits, no artificial sweeteners, no trans fats). FIG. 3B illustrates a user interface 320 where the user has selected the green Helthy G icon, which explain the rational of the method for selecting the product.

Conversely, when a product is not recommended, a different icon is presented next to the food product image, such as a yellow warning sign. FIG. 3C is a user interface 340 illustrating a product page of Noosa Yoghurt Lemon Whole Milk Yogurt Box with a yellow warning icon. Helthy can further overlay information such as text explains why the product is not recommended, like that the product has a high glycemic load and high added sugars. FIG. 3D is a user interface 360 illustrating the rationale for why the product has the warning icon (e.g., total sugars being above 5 g, having added sugars, having trans fats).

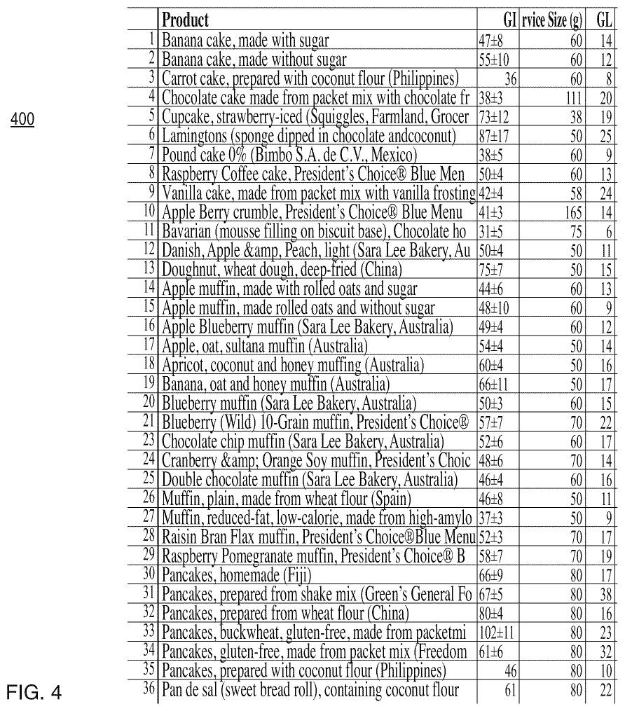

FIG. 4 is a diagram of a table or database 400 illustrating products, and various statistics associated with each product, including glycemic load. The technology for this extension is based on software methods. First, a generic method is a one-size-fit-all model. The model employs a glycemic load database of 3000 products from the University of Sydney. Glycemic load is a number associated with a given food that estimates how much the food raises a person's blood glucose level after eating it. In embodiments, glycemic load is based on glycemic index (i.e., glycemic load is the glycemic index multiplied by the amount of carbohydrates and divided by a constant such as 100). Glycemic load should be within 20 because anything 20 or over is considered high glycemic load.

The Joslin also recommends that a daily maximum of 45% total calories should come from carbs, 40% from fat, and 30% from protein. In an embodiment, the method therefore flags products that have more than 55% total calories coming from carbs, 50% coming from fat, and 40% from protein. This disclosure therefore provides a method to determine

The model is further based on variables from the Joslin Center, a non-profit and world leader in diabetes care. The method can flag products based on ingredients in the products and measurements of the product. For example, in individual products, trans-fat should always be 0. As another example, sodium listed in the nutrition facts should be <140 mg per serving. Joslin Center provides recommendations for daily meals, however there is no current method to translate these meals-based rules to individual product recommendations.

Therefore, additional sets of rules should be employed with the method. For example, Joslin Center recommends daily limits for total sugar of 25 g for women and 37.5 g for men, but provides no individual product recommendations. In an embodiment, the method therefore flags products that have more than 5 g of sugar per serving per product. After calculating flags for each individual category of nutritional fact, the method calculates a Boolean or-operation of whether any of the flags are raised, and if that result is true, the product is not recommended. If the result is false, and none of the flags are raised, then the product is recommended. In other words, a product is approved when each rule is satisfied, but the product is not suggested when any one rule is not satisfied.

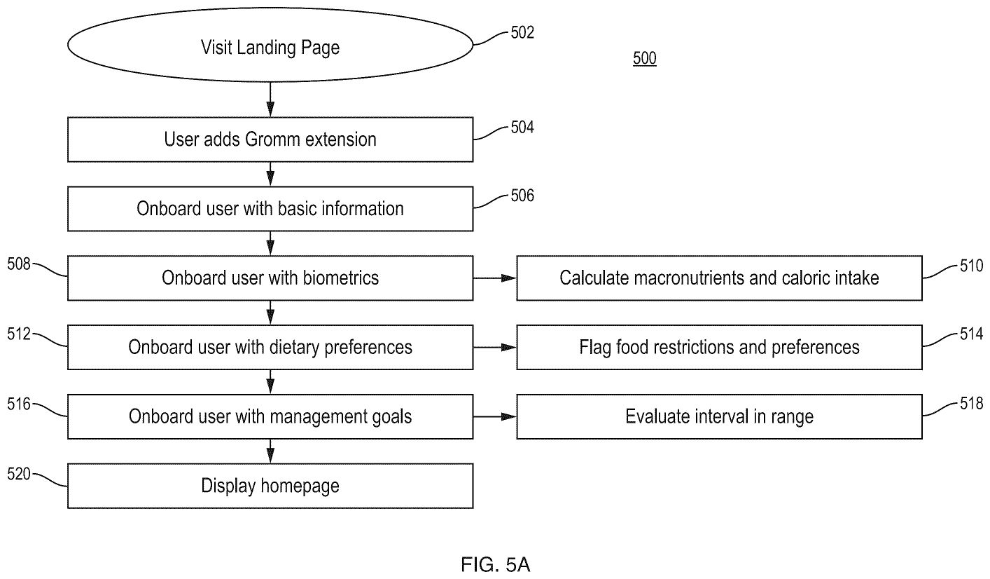

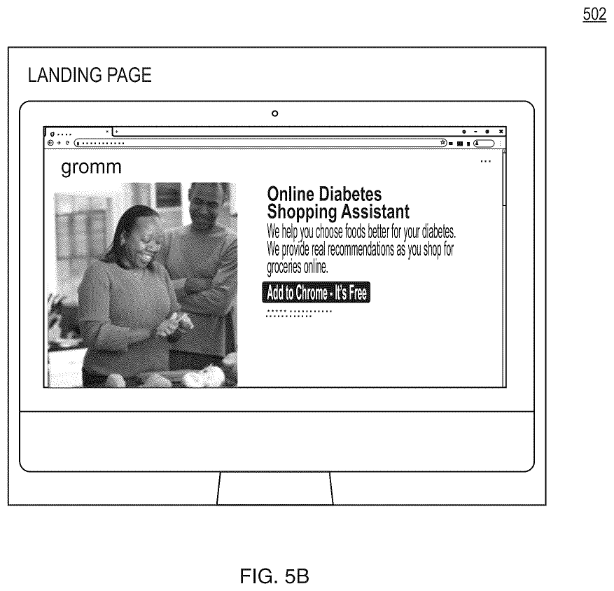

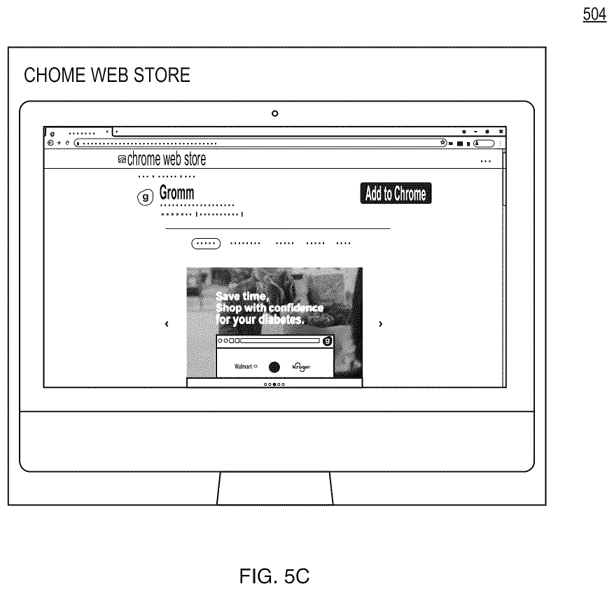

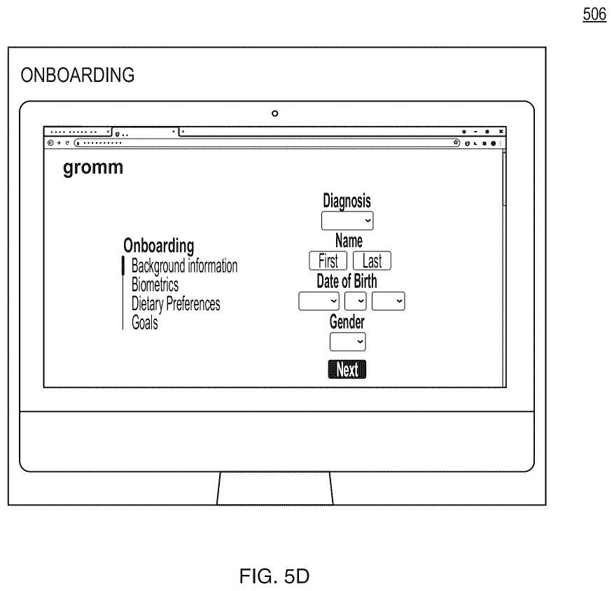

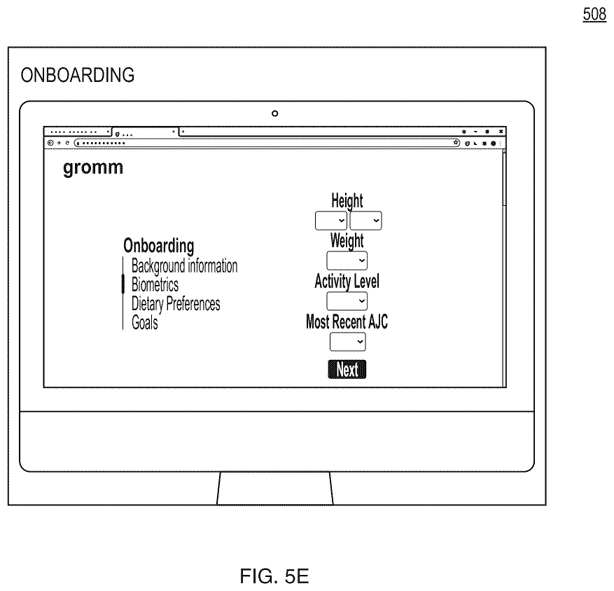

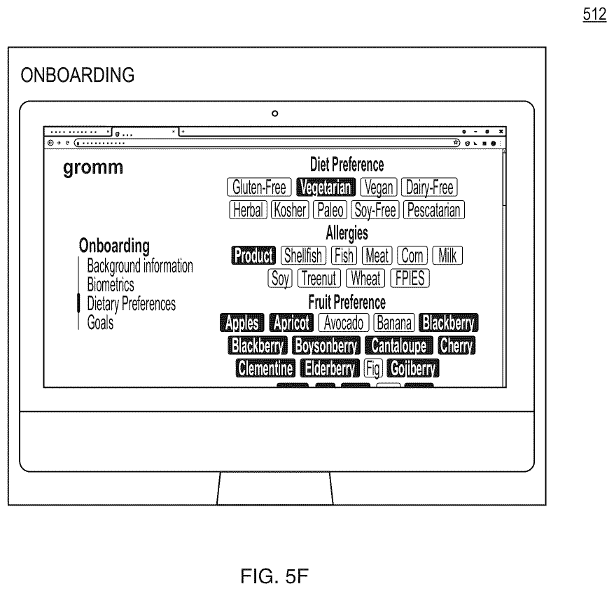

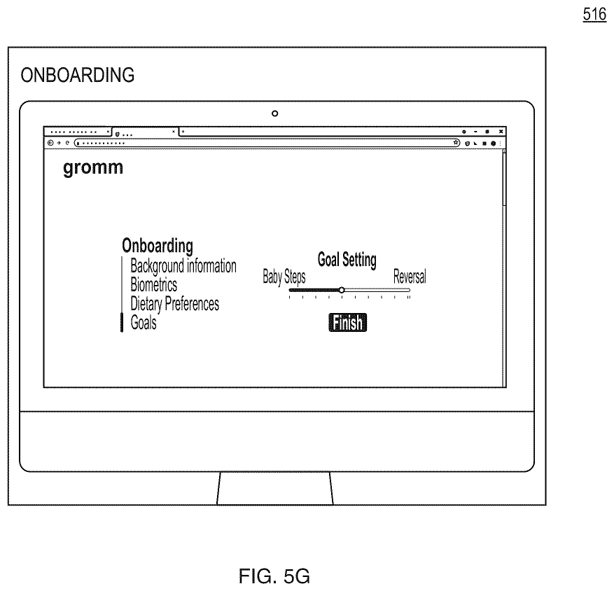

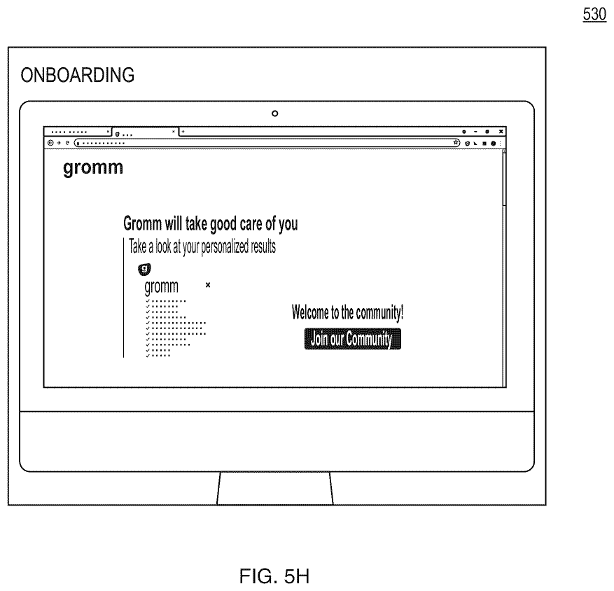

FIG. 5A is a flow diagram 500 illustrating an example embodiment of setting up the extension for the user. In an embodiment, use of the product can begin at Helthy's website or via redirection from another source. After visiting the landing page (502, FIG. 5B) and adding the extension (e.g., a Chrome extension installed in the Google™ Chrome browser) (504, FIG. 5C) Glomm's method personalizes the method to the individual (e.g., based on a profile of the user) (506, 508, 512. 516). A person of ordinary skill in the art can recognize that the browser extension can be applied to other browsers. The user can input basic profile information such as name, birthday, diagnosis, and gender (506, FIG. 5D). The user then can input biometric data such as weight, height, activity level, and hemoglobin A1C (508, FIG. 5E). The method can calculate for individual caloric intake and macronutrient needs based on the biometric data (510). The user can further input their dietary restrictions and preferences, such as vegetarian, no red meat, allergies, gluten intolerance, kosher diets, and other food preferences (512 FIG. 5F), the method can flag those ingredients and recommend products that are preferred for users (514). In addition, users can input and set management goals, such as reversing or controlling diabetes, (516, FIG. 5G), and the method can adjust limits in range based on goal setting (518). Once onboarding is complete, the extension displays the Helthy homepage (520). In some embodiments, the Helthy homepage can include a link to joining the Helthy community, as shown by user interface 530 of FIG. 5H. The Helthy community page includes diabetes education, meal ideas, and trending items.

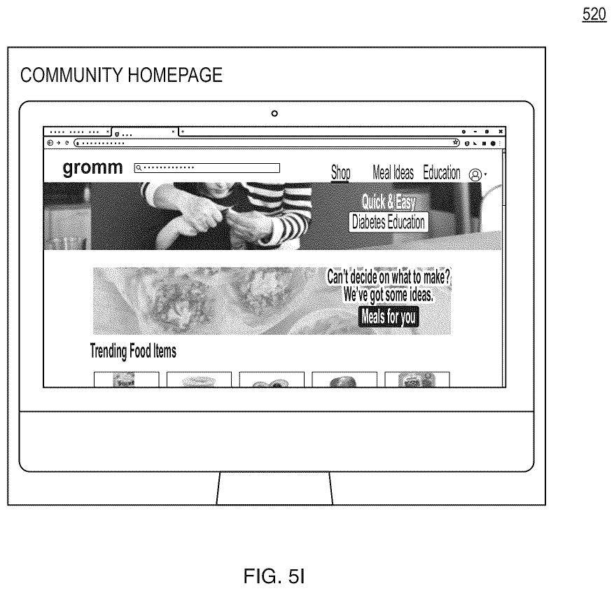

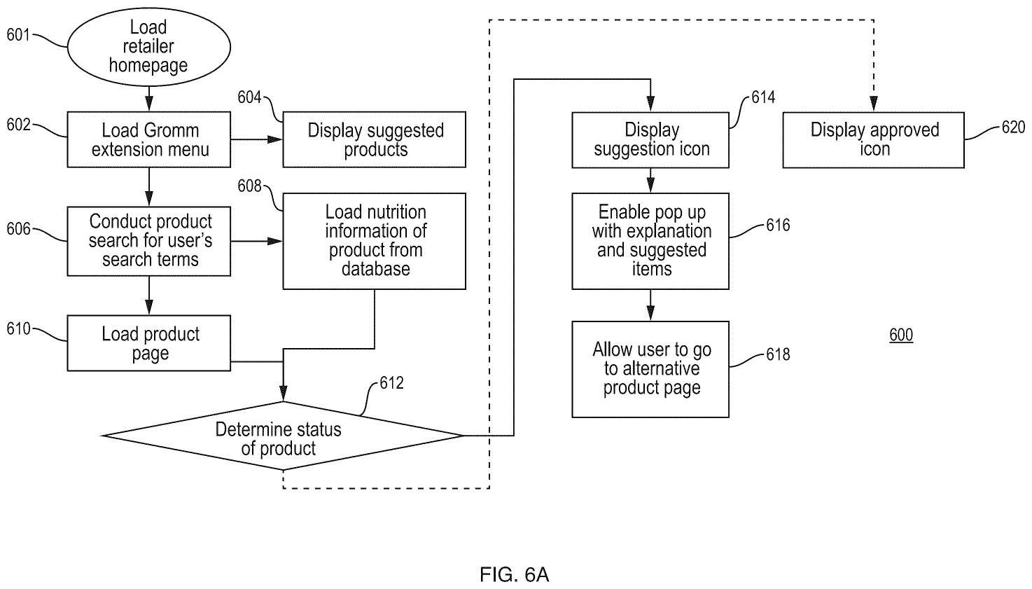

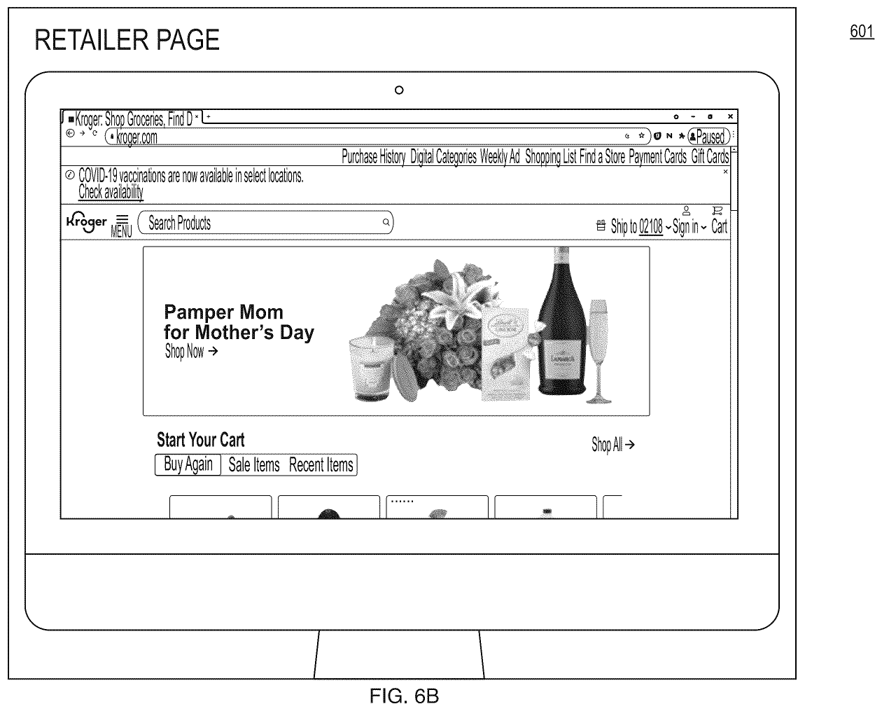

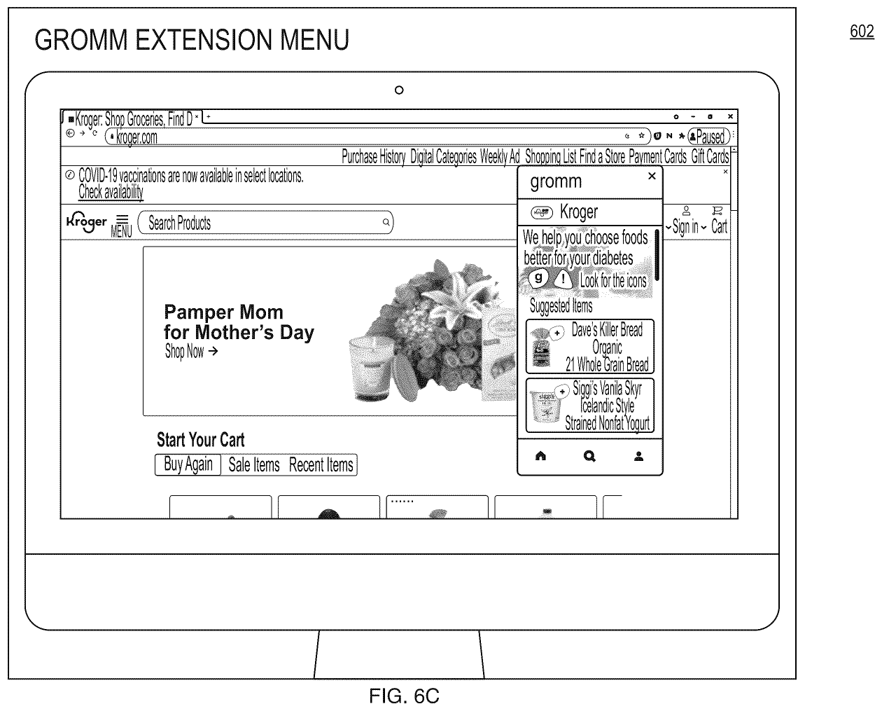

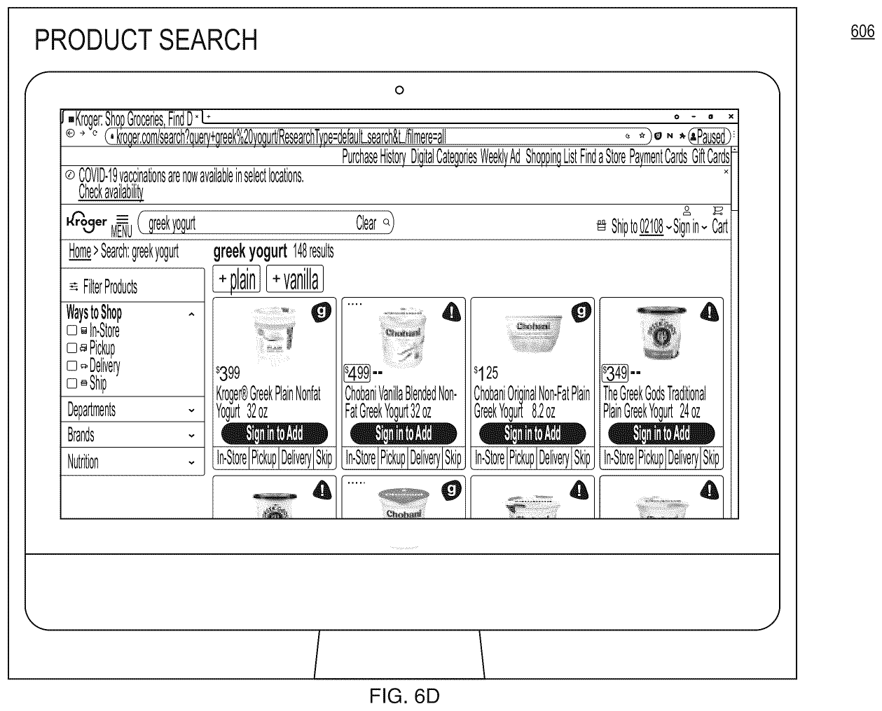

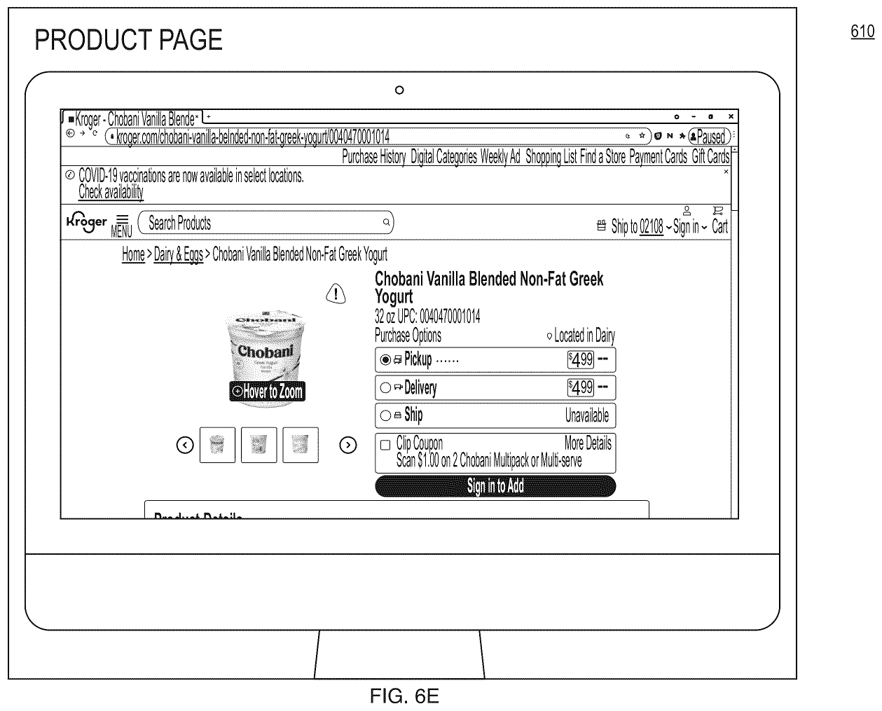

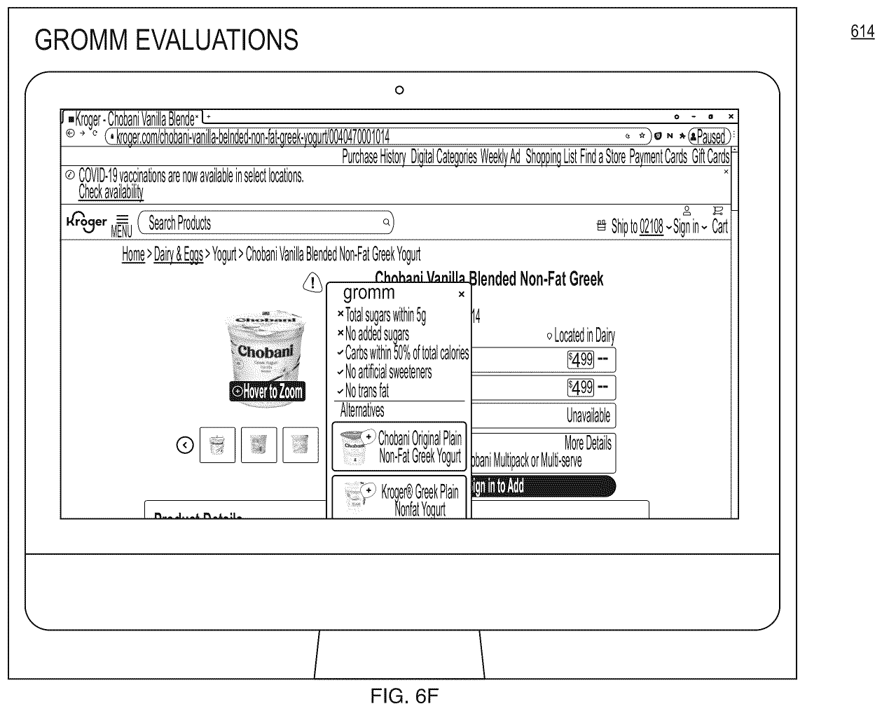

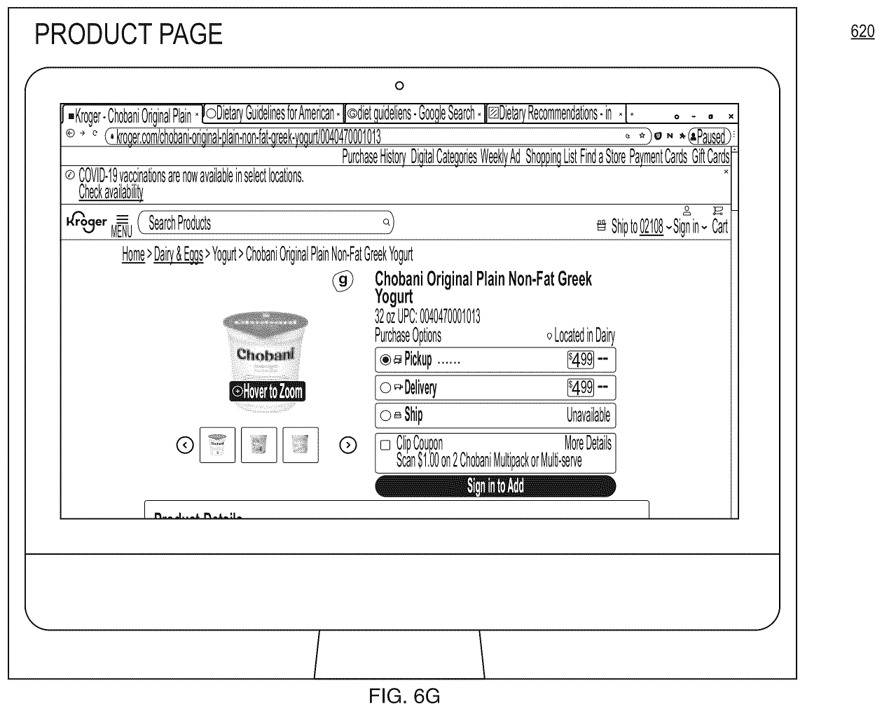

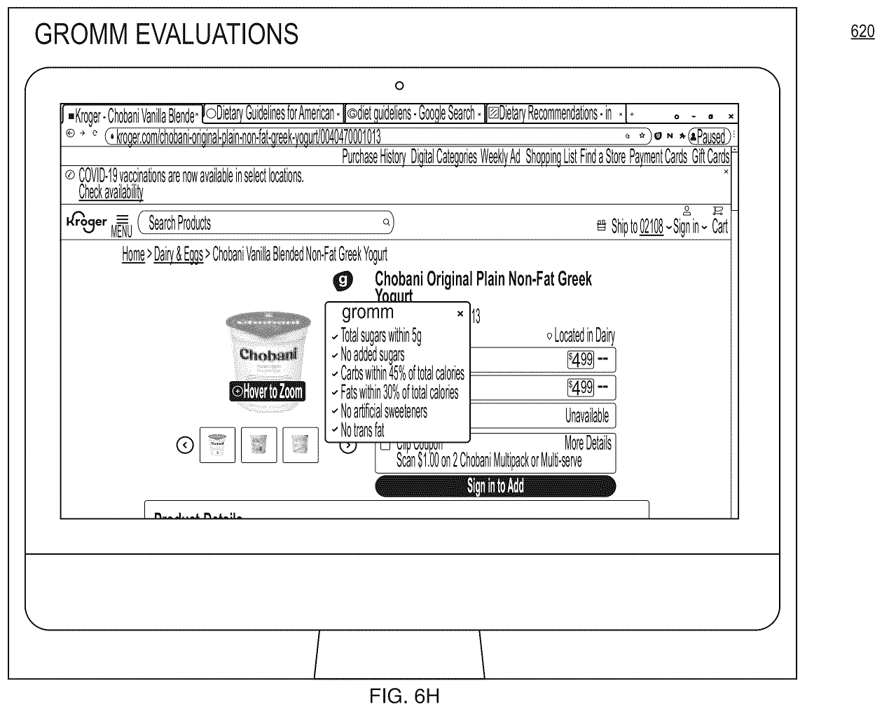

FIG. 6A is a flow diagram 600 illustrating an example embodiment of a process for using the extension once configured. After onboarding, the user can view the personalized results and see what Helthy measures. When the user wants to shop for groceries online, they can visit any retailer (e.g., Krogers) homepage in the browser with the Helthy extension installed (601, FIG. 6B). The extension window should automatically load (e.g., pop-up), to welcome the user and introduce the user to the Helthy (602, FIG. 6C). The menu also automatically suggests products good for people with diabetes based on the profile (604).

Upon the user searching for a product, such as Greek yogurt, icons appear on the resulting search result list so that the user can compare products side by side (606, FIG. 6D). Upon searching, Helthy loads nutrition information of the product from a database (608). A green icon indicates that the product is good for diabetes and a yellow icon indicates that the product may not be good for diabetes. The user can load a product page by clicking on the item in the search results, or displayed in the Helthy extension (610, FIG. 6E). The Helthy extension determines the status of the product as a suggested product or a product with a warning (612). With the example of a Chobani® vanilla Greek yogurt, the warning icon appears on the top right of the product image on the product page (614, FIG. 6F). Upon the user clicking on the icon, Helthy displays its evaluation of the product, including factors such as the total sugars per serving being above 5 g, there are other added sugars, but, the carbs are within limit and there are no artificial sweeteners or trans fats, which is good (616, FIG. 6F). Helthy also suggests, within the pop-up, alternative products that are better for diabetes, such as the Chobani original plain non-fat Greek yogurt (618). If the user selects the alternative product, Helthy displays the green G icon and Helthy lets the user know why the product is good for a patient with diabetes (620, FIG. 6G). In this case, it's because total sugars per serving are within 5 g, there are no added sugars, carbs are within 45% of total calories, fats are within 30% of total calories, there are no artificial sweeteners or trans fats (620, FIG. 6H).

The Gromm/Helthy backend method operates as follows. First, the method scans each product, either in advance or in real-time, and parses its nutrition data with dedicated functions for detecting: added sugars, refined grains, nutrition label values, trans fats, total fats (e.g., whether the fats are lower than 40% of total calories to be consumed by user), saturated fats (e.g., should be less than 15% of total calories to be consumed by user), total carbohydrates, protein, sodium). The method then considers negative factors (e.g., added sugars, added sugars, refined grains, nutrition label values, trans fats, total fats being above a threshold, saturated fats being above a threshold, total carbohydrates, sodium) and positive factors (e.g., low fat, low saturated fats, low carbohydrates, high protein), and determines a recommendation status (e.g., recommending a product when it satisfies all factors being considered).

A person of ordinary skill in the art can appreciate that Gromm or Helthy can be standalone mobile app, standalone computer application, or standalone website. With a mobile app, the user can scan a barcode, name of the product, or other identifying information of the product to see Gromm or Helthy's recommendation.

A person of ordinary skill in the art can appreciate that the above system can be implemented as an Application Programming Interface (API) that can integrate into other systems, such as the back-end of a supermarket or restaurant's website.

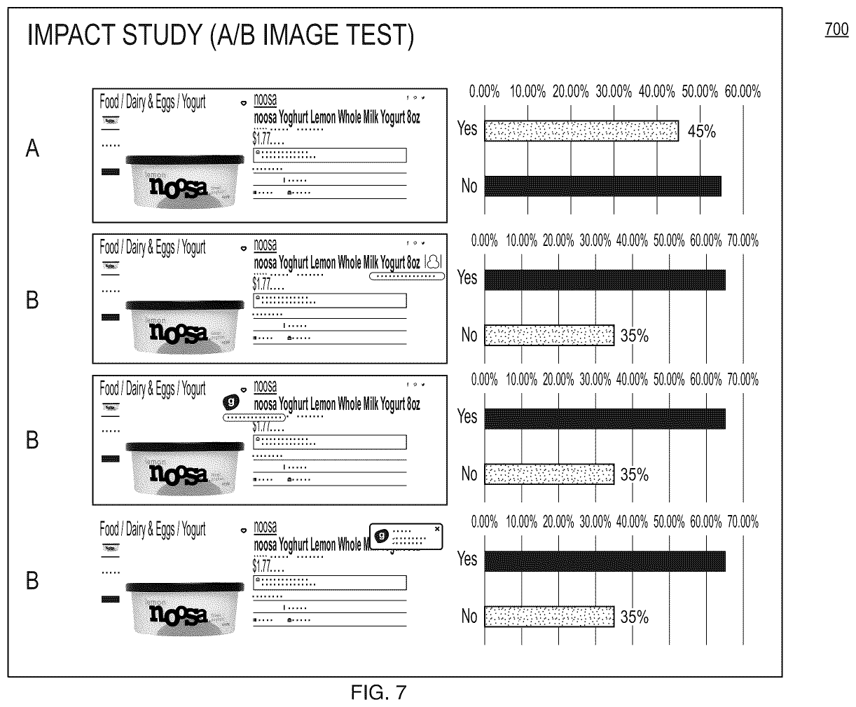

FIG. 7 is a diagram 700 illustrating a study conducted with test images, illustrating the effectiveness of the method in the real world with real patients. For image A, 45% of respondents said they would buy the product. And out of those that said yes to purchasing the product in image a, 35% then converted to not buying the product after seeing the added information. So the added information, no matter the icon and text style or placement, was able to nudge 35% of participants to not buying the product.

In addition to grocery shopping, food delivery is also on the rise, and Helthy has the potential to also vet food delivery meals.

Consumers globally are also seeking healthier food options to purchase. When clicked, Helthy displays reasoning that a product is flagged. For example, for a product that has carbohydrates are within limit and with no artificial sweeteners, the product may be flagged because there is a lot of total sugar and added sugars, and it may contains trans fats.

In addition, Helthy can propose alternative products that are healthier for the user's diabetes.

### Evaluation

In an Institutional Review Board (IRB) exempt a/b image study with 50 participants, it was sought to understand if the icons and added information nudge participants to not purchase certain products. Image A is how a user may see a product on a retailer website, and Image B include added information such as these icons and also text that says high glycemic load and high added sugars. For image A, 45% of respondants said they would buy the product. Out of those that said yes to purchasing the product in image a, 35% then converted to not buying the product after seeing the added information. Therefore, the added information, no matter the icon and text style or placement, was able to nudge 35% of participants to not buying the product.

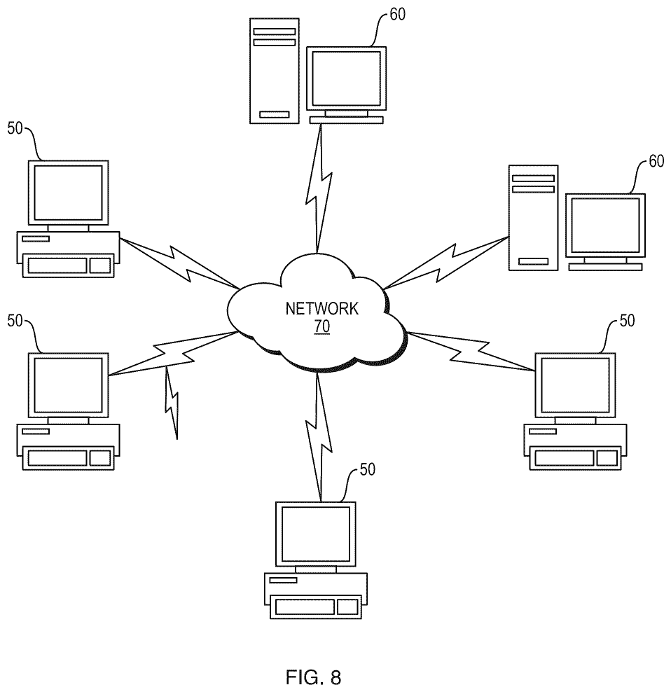

FIG. 8 illustrates a computer network or similar digital processing environment in which embodiments of the present invention may be implemented.

Client computer(s)/devices 50 and server computer(s) 60 provide processing, storage, and input/output devices executing application programs and the like. The client computer(s)/devices 50 can also be linked through communications network 70 to other computing devices, including other client devices/processes 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

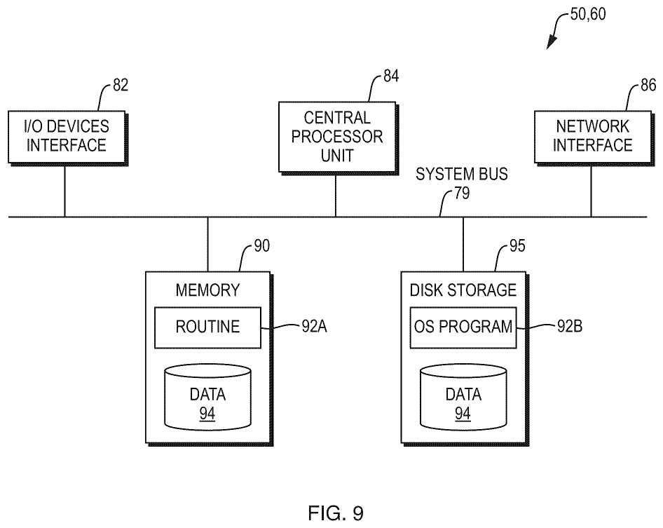

FIG. 9 is a diagram of an example internal structure of a computer (e.g., client processor/device 50 or server computers 60) in the computer system of FIG. 8. Each computer 50, 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output ports, network ports, etc.) that enables the transfer of information between the elements. Attached to the system bus 79 is an I/O device interface 82 for connecting various input and output devices (e.g., keyboard, mouse, displays, printers, speakers, etc.) to the computer 50, 60. A network interface 86 allows the computer to connect to various other devices attached to a network (e.g., network 70 of FIG. 5). Memory 90 provides volatile storage for computer software instructions 92 and data 94 used to implement an embodiment of the present invention (e.g., Helthy extension, database module detailed above). Disk storage 95 provides non-volatile storage for computer software instructions 92 and data 94 used to implement an embodiment of the present invention. A central processor unit 84 is also attached to the system bus 79 and provides for the execution of computer instructions.

In one embodiment, the processor routines 92 and data 94 are a computer program product (generally referenced 92), including a non-transitory computer-readable medium (e.g., a removable storage medium such as one or more DVD-ROM's, CD-ROM's, diskettes, tapes, etc.) that provides at least a portion of the software instructions for the invention system. The computer program product 92 can be installed by any suitable software installation procedure, as is well known in the art. In another embodiment, at least a portion of the software instructions may also be downloaded over a cable communication and/or wireless connection. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals may be employed to provide at least a portion of the software instructions for the present invention routines/program 92.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method comprising:
responsive to a user of a user device selecting a food product in a user interface of the user device, determining nutrition information of the food product;
processing the nutrition information of the food product using a dietary model, the dietary model providing a purchase recommendation and one or more recommendation reasons;
displaying, in the user interface, the purchase recommendation.

2. The method of claim 1, further comprising:
determining at least one alternative food product; and
displaying, with the purchase recommendation, the at least one alternative food product.

3. The method of claim 1, wherein determining nutrition information of the food product includes one or more of: parsing food information displayed on a retailer website, accessing a database of nutrition information of food products, and interfacing with an application programming interface of the retailer website having the nutrition information.

4. The method of claim 1, wherein the dietary model is a rule based model that factors in the nutrition information.

5. The method of claim 4, wherein the dietary model factors in nutrition information including at least one of: presence of trans-fats, amount of added sugars, amount of refined grains, amount of artificial sweeteners, amount of sodium.

6. The method of claim 4, wherein the dietary model further factors in user information.

7. The method of claim 5, wherein the dietary model calculates a daily limit for the user based on their information.

8. The method of claim 1, wherein the food product is at least one of a food product at a store, a meal kit, and a meal from a restaurant.

9. The method of claim 1, wherein the user interface is at least one of a browser with a browser extension installed, a browser connected to a retailer website, and a mobile application.

10. The method of claim 1, wherein the model is configured to provide recommendations to a user having the goals of at least one of: reversing diabetes, removing diabetes, improving general health, and weight loss.

11. A system comprising:
a processor; and
a memory with computer code instructions stored thereon, the processor and the memory, with the computer code instructions, being configured to cause the system to:
responsive to a user of a user device selecting a food product in a user interface of the user device, determine nutrition information of the food product;
process the nutrition information of the food product using a dietary model, the dietary model providing a purchase recommendation and one or more recommendation reasons;
display, in the user interface, the purchase recommendation.

12. The system of claim 11, wherein the processor is further configured to cause the system to
determine at least one alternative food product; and
display, with the purchase recommendation, the at least one alternative food product.

13. The system of claim 11, wherein determining nutrition information of the food product includes one or more of: parsing food information displayed on a retailer website, accessing a database of nutrition information of food products, and interfacing with an application programming interface of the retailer website having the nutrition information.

14. The system of claim 11, wherein the dietary model is a rule based model that factors in the nutrition information.

15. The system of claim 14, wherein the dietary model factors in nutrition information including at least one of: presence of trans-fats, amount of added sugars, amount of refined grains, amount of artificial sweeteners, amount of sodium.

16. The system of claim 14, wherein the dietary model further factors in user information.

17. The system of claim 16, wherein the dietary model calculates a daily limit for the user based on their information.

18. The system of claim 11, wherein the food product is at least one of a food product at a store, a meal kit, and a meal from a restaurant.

19. The system of claim 11, wherein the user interface is at least one of a browser with a browser extension installed, a browser connected to a retailer website, and a mobile application.

20. The system of claim 11, wherein the model is configured to provide recommendations to a user having the goals of at least one of: reversing diabetes, removing diabetes, improving general health, and weight loss.
